(19) United States
(12) Patent Application Publication
(10) Pub. No.: US 2016/0102166 A1
(43) Pub. Date: Apr. 14, 2016

(54) NATURAL OIL POLYOLS DERIVED FROM POST-CONSUMER RECYCLE OILS

(71) Applicants: Johnson Controls Technology Company, Holland, MI (US); BEIJING JOHNSON CONTROLS AUTOMOTIVE COMPONENTS CO., Beijing (CN)

(72) Inventors: Haifeng Ni, Shanghai (CN); Jenny Dong, Beijing (CN); Yao Gu, Qingdao, Shandong (CN); James T. McEvoy, Howell, MI (US); William Li, Ypsilanti, MI (US)

(73) Assignee: Johnson Controls Technology Company, Holland, MI (US)

(21) Appl. No.: 14/889,809

(22) PCT Filed: May 5, 2014
(86) PCT No.: PCT/US14/36819

§ 371 Date: Nov. 6, 2015

Related U.S. Application Data
(60) Provisional application No. 61/969,543, filed on Mar. 24, 2014.
(60) Provisional application No. 61/820,571, filed on May 7, 2013.

(51) Int. Cl. C08G 18/48; C08G 18/72; C08G 18/08; C07C 67/26; C07C 67/12

(52) U.S. Cl. CPC C08G 18/4887; C07C 67/26; C07C 67/12; C08G 18/14; C08G 18/72

(57) ABSTRACT

Disclosed embodiments include an open cell, molded polyurethane foam comprising the reaction product of a reaction mixture comprising: an isocyanate mixture; and a polyol formulation, comprising a glycerin-initiated, alkylene-oxide capped natural oil polyol having a molecular weight of between 3,000 and 8,000 and a polydispersity index (PDI) of between approximately 1.5 and 2.5, wherein the PDI is defined as the ratio of the weight-average molecular weight, Mw, to the number-average molecular weight, Mn.

### CROSS REFERENCE TO RELATED APPLICATIONS

This application claims priority from and the benefit of U.S. Provisional Application Ser. No. 61/820,571, entitled “NATURAL OIL POLYOLS DERIVED FROM POST-CONSUMER RECYCLE OILS,” filed May 7, 2013, and U.S. Provisional Application Ser. No. 61/969,543, entitled “Title of Invention: NATURAL OIL POLYOLS DERIVED FROM POSTCONSUMER RECYCLE OILS,” filed Mar. 24, 2014, both of which are hereby incorporated by reference in their entirety for all purposes.

### BACKGROUND

The present disclosure relates generally to producing polyols from recycled waste oils and using them to generate a foam product.

Polyurethanes are a general class of polymers in which organic repeating units are joined by carbamate and urea linkages. Polyurethanes are generally produced by reactions in which a polyol having two or more hydroxyl groups is reacted with an isocyanate having two or more functional isocyanate groups. The hydroxyl groups and isocyanate groups may react with one another to form carbamate and urea linkages. To facilitate these polymerization reactions, the reaction materials may be heated and, alternatively or additionally, a catalyst may be provided as well as a surfactant.

Generally, polyols include polyether moieties produced by reacting an initiator, such as dipropylene glycol, sorbitol, sucrose, or glycerin, with an extender such as propylene oxide followed by an ethylene oxide to create a more reactive species. The reactivity of the polyol may be influenced by the extender used, as the extender may determine the amount of primary and secondary hydroxyl groups (i.e., the functionality) of each polyol molecule. For example, a polyol initiator and functional additions made to the initiator will determine whether extending with propylene oxide may generate mostly secondary hydroxyl groups, whereas the use of ethylene oxide may result in a greater number of primary hydroxyl groups. The reactivity of the polyol may influence the overall properties of the polyurethane foams. For example, a high functional polyol with several hydroxyl groups may produce more rigid polyurethane foams as a result of more cross-linkages between the polyol and isocyanate. Other polyol characteristics that may affect the properties of the polyurethane foam are molecular weight, functionality, and viscosity, to name a few.

### SUMMARY

In a first embodiment, an open cell, molded foam is provided that is produced by a process. The process includes reacting a polyol formulation with an isocyanate mixture, wherein at least one component of the polyol formulation is produced by a process comprising: reacting a first polyol with an acid anhydride compound to produce a monol having two or more polycarboxylic acid substituents; reacting the monol having two or more polycarboxylic acid substituents with an epoxidized fatty acid of a post-consumer recycle oil to produce a polyol branching agent; reacting the polyol branching agent with a polyol initiator to link at least two molecules of the polyol branching agent together to produce a branched polyol; and capping the branched polyol with an alkylene oxide to produce a natural oil-based polyol having a molecular weight of between 2,000 and 6,000.

In another embodiment, a method includes reacting a first polyol with an acid anhydride compound to produce a monol having two or more polycarboxylic acid substituents; reacting the monol having two or more polycarboxylic acid substituents with an epoxidized fatty acid of a post-consumer recycle oil to produce a polyol branching agent; extracting the polyol branching agent from a reaction mixture produced from the monol having two or more polycarboxylic acid substituents and the epoxidized fatty acid of a post-consumer recycle oil using one or more solvents; reacting the extracted polyol branching agent with a polyol initiator to link at least two molecules of the polyol branching agent together to produce a branched polyol; and capping the branched polyol with an alkylene oxide to produce a natural oil-based polyol having a molecular weight of between 2,000 and 6,000.

In a further embodiment, an open cell, molded polyurethane foam includes the reaction product of a reaction mixture comprising: an isocyanate mixture; and a polyol formulation, comprising a glycerin-initiated, alkylene-oxide capped natural oil polyol having a molecular weight of between 3,000 and 8,000 and a polydispersity index (PDI) of between approximately 1.5 and 2.5, wherein the PDI is defined as the ratio of the weight-average molecular weight, Mw, to the number-average molecular weight, Mn.

### DRAWINGS

FIG. 1 is a block diagram of an embodiment of a method for treating used oils to produce a NOP.

FIG. 2 is a block diagram of an embodiment of a method for reacting a refined oil from a used oil source to produce a NOP.

FIG. 3 is a block diagram of an embodiment of a method for producing a high molecular weight NOP using a branching technique.

FIG. 4 is a combined plot of hydroxyl number and epoxy number of a natural oil polyol produced in accordance with the method of FIG. 3 as a function of reaction temperature.

FIG. 5 is a combined plot of hydroxyl number and epoxy number of a natural oil polyol produced in accordance with the method of FIG. 3 as a function of reaction time.

FIG. 6 is a first region of a proton NMR spectrum of a natural oil polyol produced according to the method of FIG. 3.

FIG. 7 is a second region of the proton NMR spectrum of the natural oil polyol represented in FIG. 6 and produced according to the method of FIG. 3.

FIG. 8 is a Fourier transform infra red (FTIR) spectrum of a natural oil polyol produced according to the method of FIG. 3.

FIG. 9 is a gel permeation chromatography (GPC) chromatogram of a natural oil polyol produced according to the method of FIG. 3.

### DETAILED DESCRIPTION

Many polyols used for the synthesis of polyurethanes are synthetic. Therefore, there has been an increased interest to develop natural oil polyols from bio-based sources to minimize dependence from petroleum products and reduce the carbon footprint. Generally, natural oil polyols (NOPs) are derived from vegetable oils, such as soy, canola, and palm, among others. These vegetable oils are derived down to their individual organic saturated and unsaturated acids from C₁₂ to C₂₂, which are used as initiators in the production of NOPs. However, the chemical processes used to synthesize the NOPs generate materials having low molecular weight and/or secondary hydroxyl groups. Moreover, sources for desired vegetable oils may be limited and/or expensive. Accordingly, it may be desirable to utilize a feedstock produced from recycled materials. In accordance with the present disclosure, such recycled materials may include used oils such as animal, cooking, fried, and waste oils. The present disclosure provides embodiments of methods for using these materials to produce NOPs, and using such NOPs to produce foams (e.g., flexible foams).

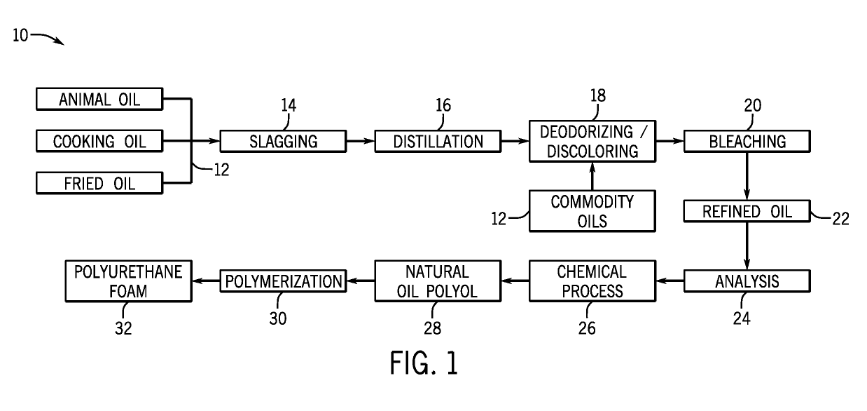

FIG. 1 is a block diagram depicting a method 10 for treating a plurality of used oils 12 to produce post-consumer recycle natural oil polyols used in the production of a polyurethane foam. As defined herein, a post-consumer recycle natural oil polyol is intended to denote a natural oil polyol obtained from a feedstock that has previously been used by a consumer (e.g., an everyday or industrial consumer). The used feedstock may then be treated according to various processes, as described herein, to produce the post-consumer natural oil polyol.

The plurality of waste oils obtained in this manner may include, but are not limited to, animal, cooking, fried, and waste oils. For example, waste oils having aliphatic chains between 8 and 22 carbon atoms such as yellow grease, tallow, lard, coconut, palm, peanut, safflower, corn, soybean, rapeseed and any other suitable bio-based spent oils may be used. The plurality of waste oils 12 undergo a variety of steps such as slagging 14, distillation 16, deodoring/discoloring 18, and bleaching 20 to produce a plurality of refined oils 22. The refined oils 22 may be modified to produce a plurality of polyhydroxylated oils. In some embodiments, the slagging 14 and skimming 16 step may be avoided such that the plurality of waste oils may be deodorized or discolored, in which processing may begin at deodoring/discoloring 18. Before the plurality of refined oils 22 can be derived into the plurality of polyhydroxylated oils, an analysis step 24 may be performed to identify property parameters, such as the relative amounts of saturated and unsaturated moieties of the individual components of the plurality of refined oils 22. In one embodiment, the analysis step 24 is used to determine how the plurality of refined oils 22 will be transformed into the plurality of polyhydroxylated oils, as the chemistry of saturated and unsaturated moieties may differ. Analysis step 24 may be done by using a variety of conventional analytical techniques and methods including, but not limited to, nuclear magnetic resonance (NMR), gas chromatography, liquid chromatography, mass spectrometry, infrared spectrometry, or UV/Vis spectrometry. Once analysis of the plurality of refined oils 22 is completed, a chemical process 26, discussed in more detail below, transforms the plurality of refined oils 22 into the plurality of post-consumer recycle natural oil polyols 28. The plurality of post-consumer recycle natural oil polyols are formulated into a polyol formulation that may include solvents, surfactants and crosslinkers, among other additives. Finally, the polyol formulation is reacted with a polyisocyanate formulation in a polymerization step 30 to produce the polyurethane foam 32.

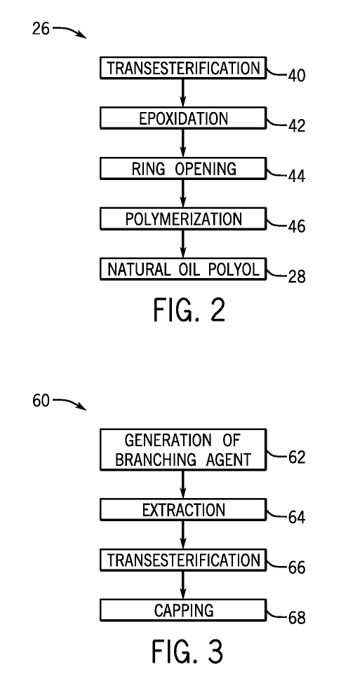

FIG. 2 illustrates one embodiment of the chemical process 26 for transforming the plurality of refined oils 22 via a series of chemical reactions into the plurality of polyhydroxylated oils. The polyhydroxylated oils are in turn polymerized into high molecular weight post-consumer recycle natural oil polyol polymers. The chemical process 26, as illustrated, includes reacting the plurality of refined oils 22 with an alcohol such that the plurality of refined oils 22 undergo transesterification to produce a plurality of modified oils (block 40). The plurality of modified oils are then oxidized to yield a plurality of epoxidized oils (block 42) and subsequently reduced to generate the plurality of polyhydroxylated oils (block 44). The plurality of polyhydroxylated oils are reacted with an extender and polymerized (block 46) to produce the plurality of post-consumer recycle natural oil polyols (block 28).

In one embodiment, desirable components of the plurality of refined oils 22 may include a plurality of triglycerides having aliphatic chains with 8 to 22 carbon atoms. More particularly, the plurality of triglycerides have aliphatic chains between 16 and 22 carbon atoms and 0-3 double bonds. For example, the plurality of triglycerides in accordance with the present disclosure may have the following formula:

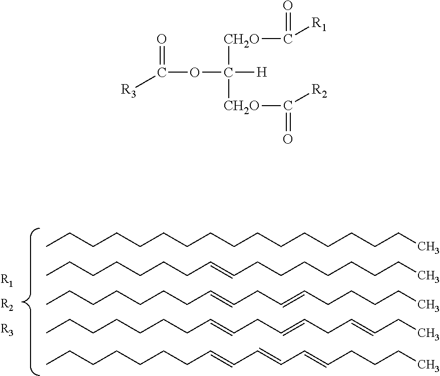

wherein the aliphatic chains, R₁, R₂, and R₃, independently include stearic, eladic, linoeladic, α-linolenic, and β-eleostearic moieties. It should be noted that the aliphatic chains are not limited and may include other aliphatic chains such as palmitic, palmitoleic, oleic, vaccenic, linoleic, erucic, arachidic, and behenic chains, among others. Moreover, the aliphatic chains R₁, R₂, and R₃ may be present in any combination. For example, in one embodiment, R₁, R₂, and R₃ may all be the same. In another embodiment, R₁, R₂, and R₃ may all be different. In yet another embodiment, R₁ and R₂ may be the same or R₁ and R₃ may be the same.

Scheme 1 below depicts one example of the transesterification of one of the plurality of refined oils 22 in accordance with block 40. For example, an unsaturated triglyceride T₁ is reacted with a glycerol G₁ at an elevated temperature. In one embodiment, the unsaturated triglyceride T₁ and the glycerol G₁ may be reacted in a 1:1 ratio. In another embodiment, the unsaturated triglyceride T₁ and the glycerol G₁ may be reacted in a ratio of 1:2. In a further embodiment, the temperature may range between 230-240° C. It should be noted, that in accordance with the present embodiments, the reactants may include other alcohols, such as sucrose, pentaerythritol, and short chain alkyl alcohols to include methanol, ethanol, and propanol or any suitable combination of alcohols.

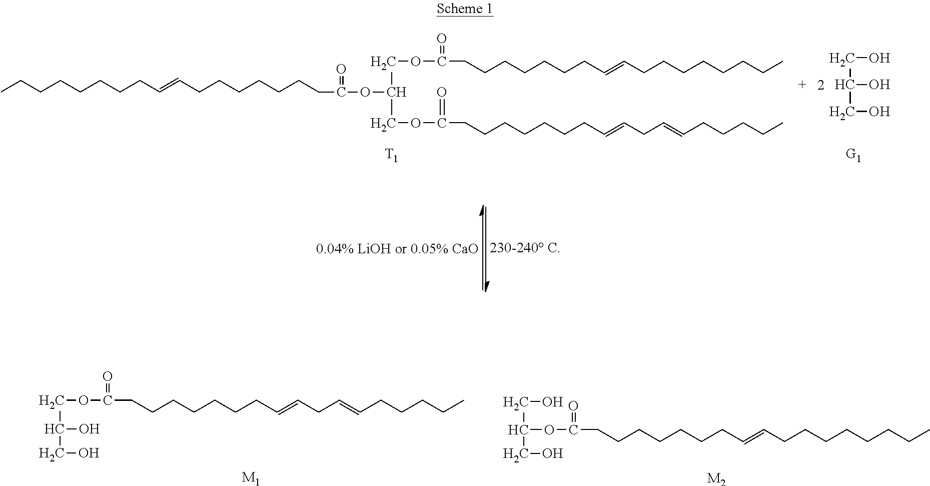

In one embodiment, a base catalyzed transesterification yields the plurality of modified oils to include a first unsaturated monoglyceride M₁ and a second unsaturated monoglyceride M₂. The transesterification may be catalyzed by metal hydroxides, metal oxides, alkoxides, carbonates, amines, organolithium agents, phase transfer catalysts, non-nucleophilic bases or any other suitable catalyst. For example, the catalyst may include, among others, sodium hydroxide, potassium hydroxide, lithium hydroxide, sodium methoxide, calcium oxide, or any combination thereof. In one embodiment, the transesterification may be acid catalyzed using any suitable acid such as a Brønsted Lowery acid to include, but not limited to hydrogen chloride or sulfuric acid. In another embodiment an enzyme such as lipase may be used to catalyze the transesterification of the plurality of refined oils 22.

It should be noted that the first and second unsaturated monoglycerides, M₁ and M₂, may be subjected to any suitable purification method known to remove any undesirable reaction by-products. For example, the purification methods may include, but are not limited to, distillation, chromatography, extraction, filtration, or a combination thereof.

After transesterification of the plurality of refined oils 22 according to block 40, as noted above, the plurality of modified oils, for example the first and second unsaturated monoglycerides, M₁ and M₂, are functionalized in a series of chemical reactions to produce the plurality of polyhydroxylated oils. For example, Scheme 2 below illustrates an oxidation reaction of the first and second unsaturated monoglycerides, M₁ and M₂, in accordance with block 42. While any epoxidation method may be used, in one embodiment the epoxidation is performed using hydrogen peroxide in glacial acetic acid at elevated temperatures. In other embodiments, the epoxidation may be performed using other oxidizing agents including, but not limited to, alkylhydroperoxides (e.g., t-butylhydroperoxide), meta-chloroperbenzoic acid (mCPBA), dioxiranes, permanganates, or other suitable oxidizing agents. In one embodiment, the first and second unsaturated monoglycerides, M₁ and M₂, are reacted with 30% hydrogen peroxide and glacial acetic acid at in a ratio of 1:1.05:0.25, respectively, at ambient temperature to produce a first epoxidized oil E₁ and a second epoxidized oil E₂. In one embodiment, the temperature may be between 60-100° C. In another embodiment, the temperature may be between 70-80° C. After separation and purification using any suitable purification technique (e.g., distillation, chromatography), the first and second epoxidized oils, E₁ and E₂, are reduced down to the plurality of polyhydroxylated oils.

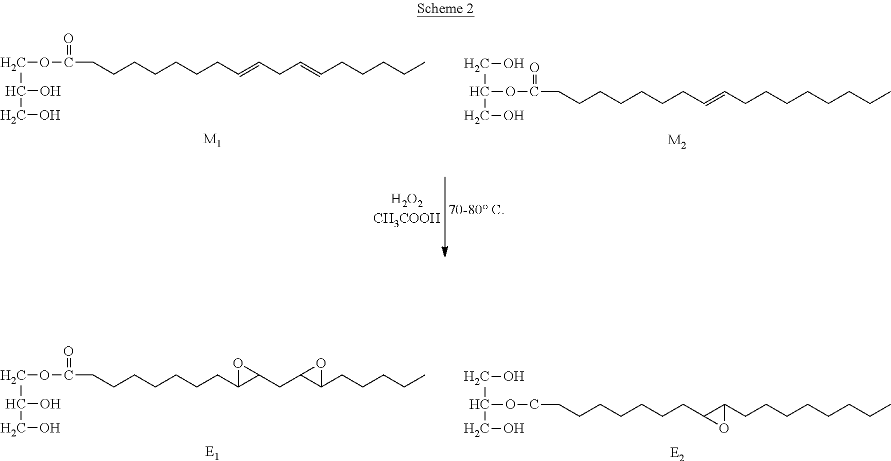

Moving now to the ring opening performed in accordance with block 44, the first and second epoxidized oils, E₁ and E₂, are reduced to generate a first polyhydroxylated oil P₁ and a second polyhydroxylated oil P₂. The first and second epoxidized oils, E₁ and E₂, are reacted with an alcohol/water mixture and undergo alcoholysis. In one embodiment, as shown is Scheme 3 below, the alcoholysis is performed at an elevated temperature and pressure using methanol to produce the first polyhydroxylated oil P₁ and the second polyhydroxylated oil P₂ having mostly secondary hydroxyl groups. The use of methanol or other short chain alcohols may be desirable to avoid steric hinderance at the secondary alcohol moieties.

In one embodiment, the temperature may be between 70-150° C. In another embodiment, the temperature may be between 80-130° C. In yet a further embodiment, the pressure may be between 0.3-0.5 MPa.

The alcohol/water mixture may include a ratio of alcohol:water of between 0.8:1-5:1. In one embodiment, the alcohol/water mixture may include a ratio of between 3:1 and 5:1. The alcohol may be an alkyl alcohol including, among others, methanol, ethanol, propanol, butanol, or the like. In another embodiment, the reduction can be carried out by an acid such as, but not limited to, lactic acid and acetic acid. In yet another embodiment, the reduction can be done with glycols, such as, but not limited to, ethylene glycol.

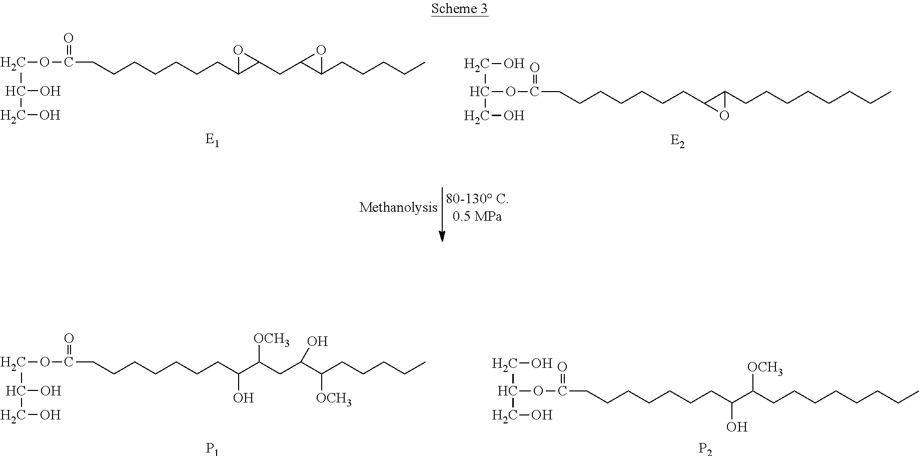

Polymerization of the first and second polyhydroxylated oils, P₁ and P₂, in accordance to block 46, may be achieved by reacting with ethylene oxide and/or propylene oxide in the presence of an acid catalyst, as shown in Scheme 4 below. In one embodiment, the polymerization is performed at a temperature between 35-40° C. In another embodiment, the polymerization may be performed at a pressure of between 0.3-0.5 MPa to produce a first post-consumer recycle natural oil polyol N₁ and a second post-consumer recycle natural oil polyol N₂, having a molecular weight of between 1,000 and 10,000, such as between approximately 3,000 and 8,000, or between approximately 4,000 and 6,000. The acid catalyst may include any suitable Lewis acid or Brønsted Lowery acid such as, but not limited to, tetrafluroboric acid, hydrochloric acid or any other similar catalyst. In another embodiment, the polymerization reaction may be base catalyzed. By way of example, the base catalyst may include potassium hydroxide, sodium hydroxide, sodium carbonate, or any other similar catalyst.

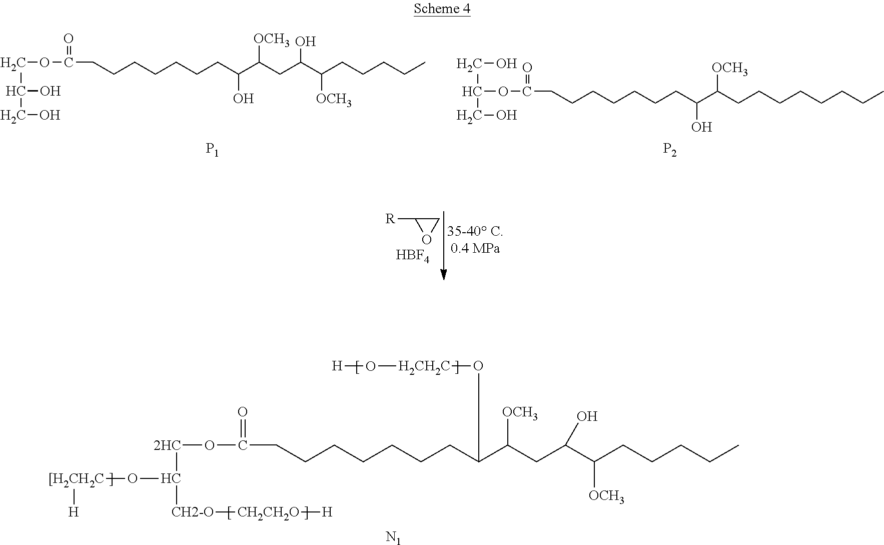

Generally, the polyols have two or more hydroxyl moieties. The average number of hydroxyl moieties per polyol molecule is generally referred to as a functionality of the natural oil polyols. In accordance with present embodiments, the post-consumer recycle NOPs may have a functionality ranging from approximately 1.1 to 8, 1.4 to 4, or 1.5 to 3. In some embodiments, a functionality of between 1.5 to 3 may be desirable to generate a foam having desired transmissivity. Such functionality may be derived from 8, 6, 4, 3, 2, and/or 1 functional materials that come to a ratio of 3.

In another embodiment, the post-consumer recycle natural oil polyols may be catalyzed using a dimetal catalyst (DMC). Scheme 5 below is a representative example of a reaction of the first polyhydroxylated oil P₁ and the second polyhydroxylated oil P₂, with propylene or ethylene oxide in the presence of a DMC that generates the post-consumer recycle natural oil polyols, such as post-consumer recycle natural oil polyol N₂. The post-consumer recycle natural oil polyols may have an average molecular weight (M_{w}) of between 1.0×10³ and 4.0×10³. In one embodiment, the post-consumer recycle natural oil polyols may have a M_{w} of between 1.6×10³ and 3.6×10³. However, it should be noted that the M_{w} may be as high as 1×10⁴.

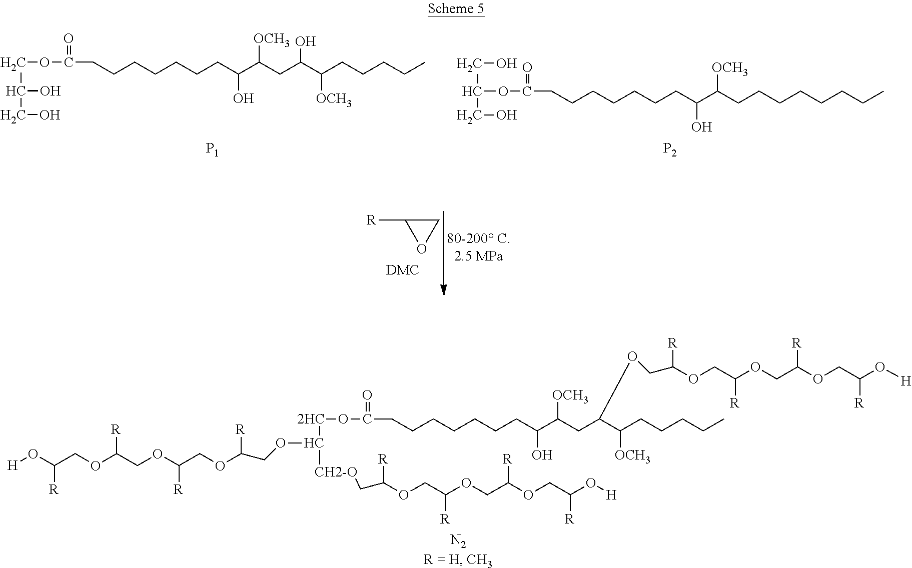

Addition of the DMC allows controlled polymerization and higher molecular weights can be achieved compared to non-catalyzed polymerizations. Furthermore, the DMC decreases monol content resulting in increased overall yield and performance of the post-consumer recycle natural oil polyols compared to natural oil polyols produced from non-catalyzed polymerizations. Reducing the monol content may be desirable, at least because the monol may act as a terminator during the polymerization of the natural oil polyols and polyisocyanate source in the production of polyurethane foams. More specifically, the presence of monol can increase the difficulty associated with achieving polyurethane foams having high molecular weight (e.g., greater than approximately 3,000). The DMC includes, but is not limited to, a metal cyanide catalyst, such as that produced by the reaction of a hexacyanocobalt salt and zinc chloride and having the following formula:

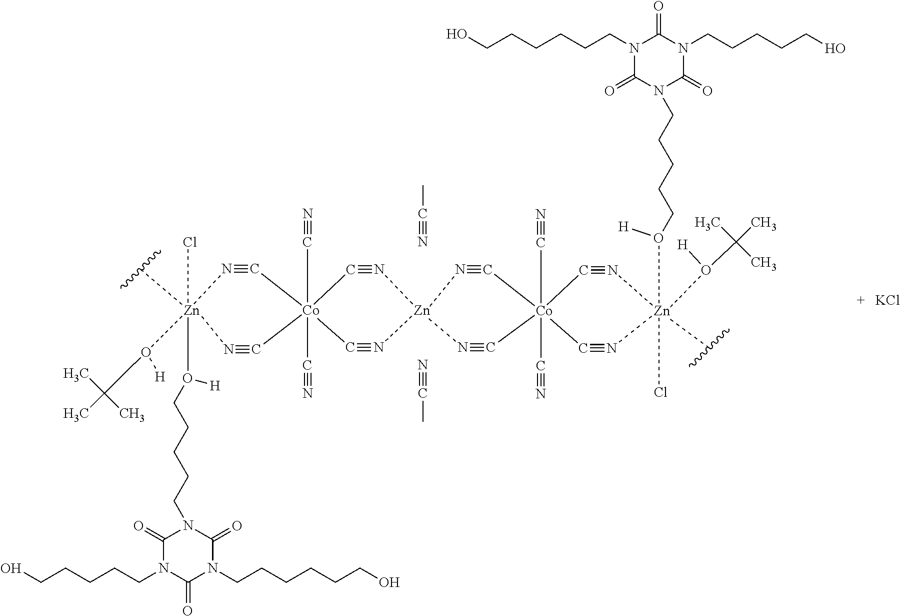

In yet another embodiment, the first polyhydroxylated oil P₁ and the second polyhydroxylated oil P₂ may be reacted with an anhydride to yield a plurality of carboxylic acid terminated monoglycerides. In one embodiment, shown in Scheme 6, the first and second polyhydroxylated oils, P₁ and P₂, are reacted with maleic anhydride at an elevated temperature and pressure to produce a first carboxylic acid terminated monoglyceride C₁ and a second carboxylic acid terminated monoglyceride C₂. In one embodiment, the temperature may be between 70-200° C. In another embodiment, the temperature may be between 90-140° C. In a further embodiment, the pressure may be between 0.4-0.8 MPa. The carboxylic acid terminated monoglycerides are further reacted with ethylene or propylene oxide to yield the post-consumer recycle natural oil polyols.

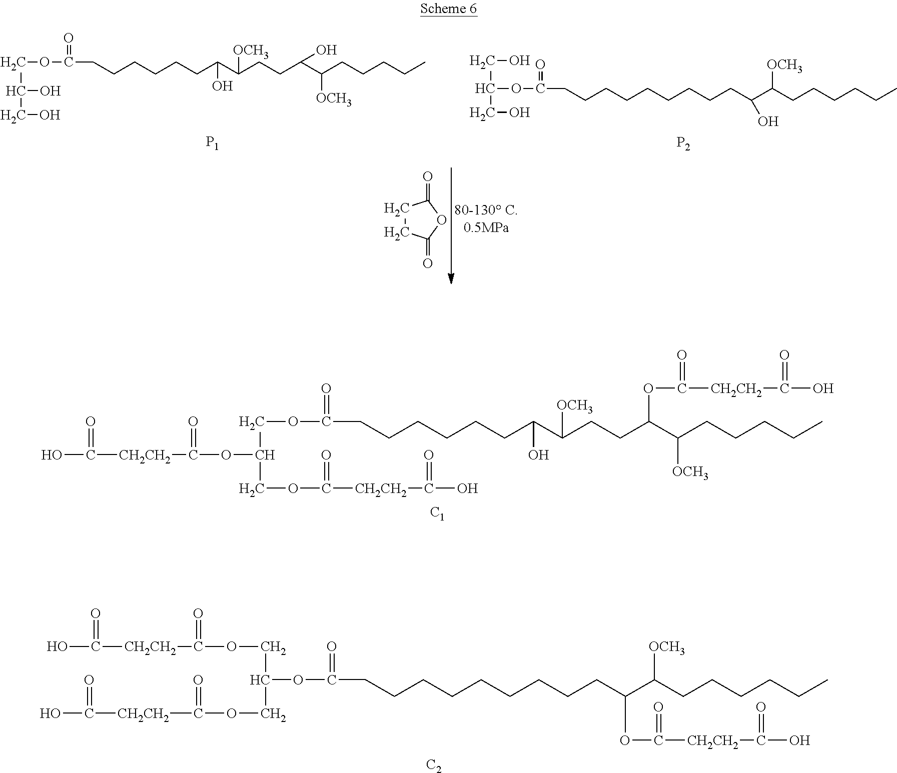

It should be noted that the reactions discussed above for the generation of post consumer recycle natural oil polyols are also applicable to refined oils 22 having only saturated aliphatic chains. In one embodiment, the saturated refined oils 22 are derived into a plurality of saturated diglycerides that are reacted with an anhydride to produce a plurality of monocarboxylated diglycerides. The anhydride includes, but is not limited to, non-cyclic or cyclic anhydrides such as succinic anhydride, maleic anhydride, or the like. Polymerization of the plurality of monocarboxylated diglycerides with ethylene and/or propylene oxide, as described above, is performed to generate a plurality of monohydroxylated diglycerides comprising primary and/or secondary alcohols.

The molecular weight of the post-consumer recycle natural oil polyols may be controlled by variation in a number of polymerization conditions. For example, varying extender concentration, catalyst composition, reaction temperature, reaction pressure, or any combination thereof, may affect the molecular weight obtained. In one embodiment, the polymerization is performed at a constant temperature and concentration of DMC and the polyhydroxylated oil and varying the concentration of the extender by a factor of between 0.7-0.8. As such, the polymerization yields post-consumer recycle natural oil polyols having an average molecular weight (M_{w}) of 1.67×10⁴. In another embodiment, the molecular weight of the post-consumer recycle natural oil polyols may be controlled by variation in the reaction temperature. As one example, at a constant concentration of DMC, extender, and the polyhydroxylated oil, increasing the temperature from 96° C. to 156° C. yields an average M_{w} of 2.36×10⁴.

Additionally or alternatively, the chemical process 26 may be modified such that different types of reactants are used for certain of the reactions (e.g., according to blocks 40, 42, 44, 46) in order to achieve variations in molecular weight (e.g., greater molecular weights) for the NOP. For example, the reactants used for transesterification according to block 40 and/or the reactants used during the ring opening according to block 44 may be different than the alcohols noted above. Because of this, additional or alternative reactions may be performed compared to the reaction sequences discussed above. Indeed, the chemical process 26 described above is not intended to be limited to the steps discussed above, or the order discussed above. Rather, the present disclosure is intended to encompass any and all permutations of these reactions, including performing these reactions in any order, in any combination (e.g., in a one-pot synthesis or sub-combinations of reactions in one pot), and in combination with other reactions.

For example, discussed below is an approach to producing NOPs that may be used in addition to the chemical process 26 described above, as a part of the chemical process 26 described above, or in lieu of the chemical process 26 described above. The approach includes using particular types of reagents, which may be referred to as branching agents, to produce NOPs at relatively higher molecular weights (e.g., above 1,000, above 2,000, or above 3,000, such as between 1,000 and 10,000, between 1,500 and 8,000, between 2,000 and 6,000, or the like) and at polydispersity levels that are much lower than would otherwise be obtained. For example, the present approaches described below may produce NOPs at a molecular weight between approximately 2,000 and 6,000 at a polydispersity index (PDI) of between approximately 1.0 and 4.0, such as between approximately 1.2 and 3.8, between approximately 1.5 and 3.5, or between approximately 1.5 and 2.5, where the PDI is defined as the ratio of the weight-average molecular weight, Mw, to the number-average molecular weight, Mn, (i.e., Mw/Mn).

In accordance with the present approaches, NOPs having these high molecular weights and relatively low polydispersities may be obtained by reacting one or more of the intermediates set forth above for the chemical process 26 with branching agents, or mixtures of branching agents, having a relatively high molecular weight (e.g., compared to a short chain alcohol, glycerol, glycerin, sucrose, or the like). For example, the branching agents may have a molecular weight that is at least 1% of the molecular weight of the NOPs having a molecular weight between approximately 1,000 and 10,000, such as between approximately 10% and 60% of the molecular weight of the NOP, between approximately 20% and 55% of the molecular weight of the NOP, or between approximately 25% and 50% of the molecular weight of the NOP. The percentages noted above for the branching agents may be a percentage of the NOP molecular weight range of between 1,000 and 10,000, between 1,500 and 8,000, between 2,000 and 6,000, or between 3,000 and 8,000.

Because of this relatively large contribution of the molecular weight of the branching agent to the overall molecular weight of the NOPs, it is possible to tailor, with a great degree of control, the molecular weight of the NOP to a desired or designed value (e.g., which may be represented by the PDI). Indeed, in certain embodiments, the molecular weight of the branching agent may be chosen so as to provide a certain molecular weight for the NOP, which may in turn be chosen to produce a foam (e.g., a flexible, molded, open cell foam) having desired characteristics. Accordingly, the properties of the foam that are ultimately obtained may depend largely on the structure of the branching agents described herein used to produce the NOPs, as well as the efficiency of their associated reactions.

Turning now to specific examples of reaction sequences and their associated branching agents, one approach in accordance with present embodiments includes using a branching agent that, when reacted with an epoxide in the ring opening process performed in accordance with block 44, produces a molecule having a molecular weight between 10% and 50% of the molecular weight of the NOP (e.g., P₅ discussed below). Because the molecular weight of the resulting molecule is relatively high, certain other processes, such as the polymerization performed according to block 46, may be reduced (e.g., the degree of polymerization may be reduced) or may be altogether replaced or not performed.

FIG. 3 depicts a process flow diagram of an embodiment of a chemical process 60 for producing NOPs, which may be performed in addition to all or a part of the chemical process 26 set forth with respect to FIG. 2, or may be performed as an alternative to the chemical process 26 discussed with respect FIG. 2. Indeed, one or more of the steps of the chemical process 60 discussed below may be performed in combination with any one or a combination of the steps of the process 26.

As illustrated, the process 60 includes generating a branching agent (block 62). The acts represented by block 62 may include any suitable reaction or set of reactions (e.g., one or more reactions) that produce an intermediate product capable of reacting with epoxidized fatty acid esters produced from a natural oil (e.g., in a second reaction), such as an epoxidized fatty acid ester produced by the acts according to block 42 of process 26 (FIG. 2). For example, the reaction or reactions may produce a polyol, a monol, a carboxylic acid-containing molecule, a polycarboxylic acid-containing molecule, a molecule having one or more hydroxyls and one or more carboxylic acids, or any other similar molecules having one or more oxygen-containing moieties. An example reaction is presented below in Scheme 7, which includes a reaction between a polyether polyol (denoted as P₃) and a carboxylic acid-containing aromatic reagent (denoted as C₂) that are combined in the presence of a catalyst (e.g., triphenylphosphine, triethylenediamine, phosphoric acid, or any combination thereof) to produce a diester functionalized monol D₁.

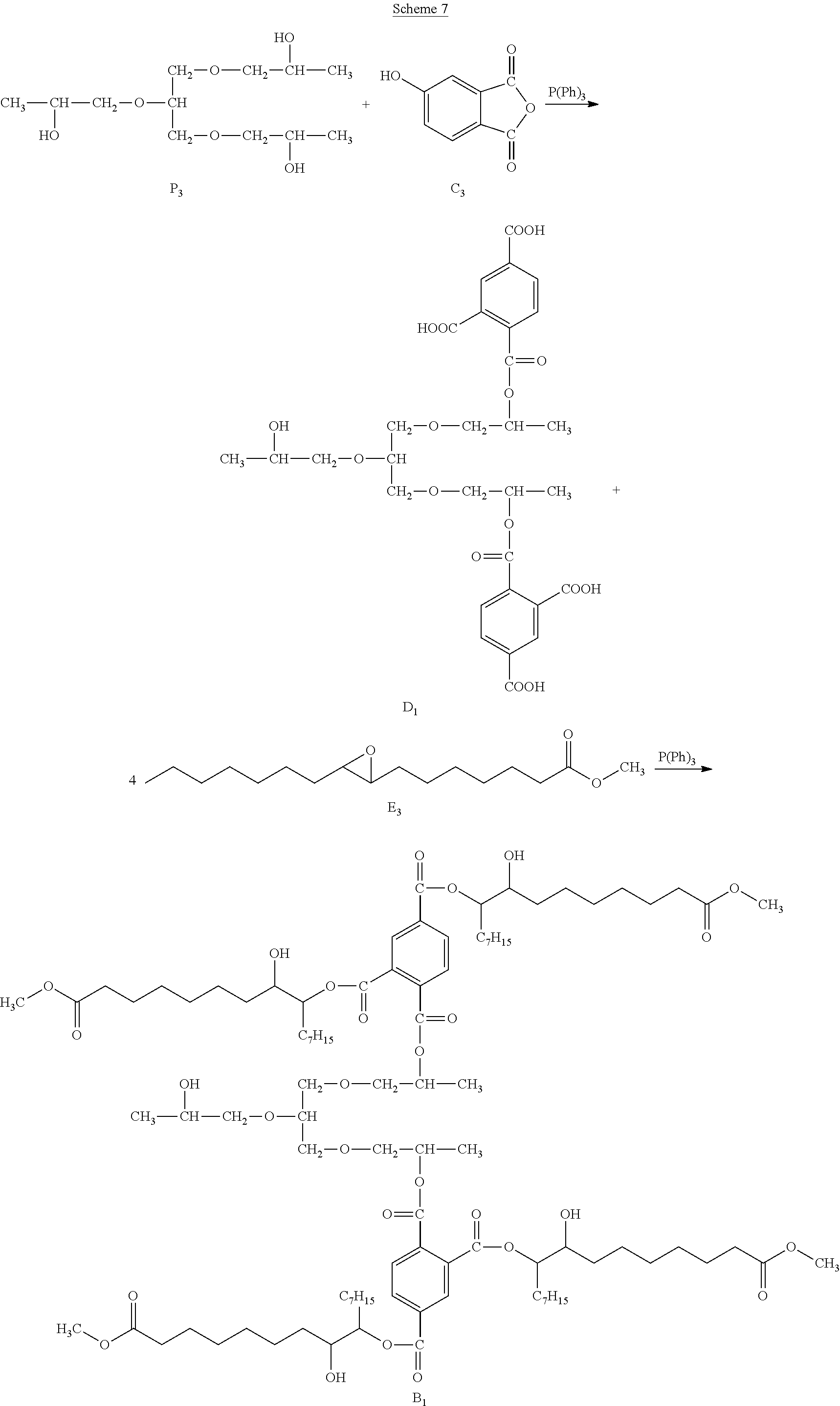

In Scheme 7, P₃ is shown as a polyether polyol having three secondary hydroxyls and three ether linkages, and is the main constituent in GY-250 polyether polyol available from Kukdo Chemical (Kunshan) Co., LTD of China. However, it should be noted the specific molecule for P₃ set forth in Scheme 7 is an example, and other polyether polyols are also presently contemplated. Example polyether polyols that may be used as P₃ may include, by way of non-limiting example, a polyether polyol having between 4 and 20 carbons, such as between 4 and 16 carbons or between 6 and 14 carbons, and between 2 and 12 oxygen atoms, such as between 2 and 10 oxygen atoms or between 4 and 8 oxygen atoms. The carbon atoms and the oxygen atoms may be linked to form one or more ether linkages and at least two hydroxyl functionalities, such as between 2 and 8 hydroxyl functionalities. Specific examples include, but are not limited to, glycols (e.g., ethylene glycol, propylene glycol), glycerin, and sucrose, among others. As a further example, the polyol P₃ may be a glycerin-initiated triol based on propylene oxide and having a molecular weight as set forth below. However, it should be noted that these materials are merely examples, and are not intended to limit the scope of the present disclosure. Indeed, the present approaches are intended to encompass any number of carbon atoms and oxygen atoms linked in any manner to form a polyether polyol.

Generally, the polyol P₃ may have a molecular weight that is between 100 and 400, such as between 200 and 300. By way of further example, the molecular weight may be between 220 and 280, such as between 230 and 270. It should be noted that it may be desirable for the molecular weight to be within these ranges to avoid or reduce steric hindrance at the secondary hydroxyl reaction sites. Indeed, as set forth in the reaction scheme 7, the reaction may largely produce the diester monol derivative of the polyether polyol P₃, as opposed to the mono-ester or the tri-ester. The presence of the monol may be used as a handle for further chemical modification, as discussed in further detail below.

Scheme 7 above also depicts the C₂ constituent of the reaction as including trimellitic anhydride. However, C₂ may include other carboxylic acid-containing reagents, such as molecules containing one, two, three, four, or more carboxylic acid and/or carboxylic acid derivatives (e.g., anhydrides, esters). Larger reactants such as these may be desirable in order to provide, among other things, a larger molecular weight and certain desirable physical properties in the foam produced using the NOP that is ultimately isolated in the present process. Furthermore, synthesizing such carboxylic-acid containing molecules may enable the tailoring of the NOP molecular weight with a much higher degree of precision than would be obtained otherwise. For example, the carboxylic acid-containing reagents may include, but are not limited to, an alkane-based carboxylic acid, a cycloalkane-based carboxylic acid, an alkene-based carboxylic acid, an aromatic-based carboxylic acid, or any combination thereof. In certain embodiments, C₂ may include a benzoic acid derivative, a phthalic acid derivative, a terephthalic acid derivative, a mellitic acid derivative, a trimellitic acid derivative, or the like. In some embodiments, it may be desirable for the carboxylic acid C₂ to be a multi-substituted aromatic molecule to enable controlled branching of the polyol (e.g., due to the planar structure of the aromatic moiety).

The reaction represented in Scheme 7 ultimately utilizes 2 equivalents of the trimellitic anhydride for every 1 equivalent of the polyether polyol. However, in some embodiments, the reaction may be performed in a range of mol ratios.

By way of further example, the weight ratio of P₃ to C₂ may be at least approximately 1:5 by weight, such as at least 1:4 by weight, or at least 1:3 by weight. By way of further example, the weight ratio of P₃ to C₂ may be between 1:5 and 1:1 by weight, such as between 1:5 and 1:2 by weight, between 1:4 and 1:2 by weight, or between 1:3 and 1:1 by weight.

The reaction between P₃ and C₂ may be promoted or catalyzed using one or more promoters/catalysts. By way of example, the reaction between P₃ and C₂ may be facilitated by an amine catalyst (e.g., a trialkyl amine catalyst), a diamine catalyst (e.g., a cyclic diamine and/or a trialkyl diamine such as triethylenediamine), a phosphorous compound (e.g., phosphoric acid, a tri-alkyl or tri-aryl phosphine such as triphenylphosphine), or similar oxophile, or any other acid (e.g., Lewis acid and/or Brønstead-Lowry acid) or similar catalyst that is capable of facilitating esterification, or any combination of such catalysts.

As noted above, the reaction between P₃ and C₂ may, in certain embodiments, produce the diester functionalized monol D₁. The diester functionalized monol D₁ may, in some embodiments, represent the major product produced via the reaction between P₃ and C₂. However, it should be noted that the relative ratios between P₃ and C₂, the particular identities of P₃ and C₂, as well as the catalyst used for their reaction, may affect the abundance of D₁ relative to other potential products, such as a mono-ester monol, or a tri-ester monol.

The diester functionalized monol D₁ may be further reacted with an epoxidized oil (e.g., epoxidized oils E₁ or E₂ noted above), represented in Scheme 7 as E₃, which, in some embodiments, has been isolated from a used oil, such as a cooking oil (and is therefore a post-consumer or post-industrial recycle oil). In one embodiment, E₃ may include a stearic, elaidic, linoleic, α-linolenic, or α-eleostearic substituent. E₃ may be substantially the same as the epoxidized oils E₁ or E₂ described above, or may be an entirely different structure. Indeed, any one or a combination of the materials discussed above, or may be any fatty acid alkyl ester (e.g., a fatty acid methyl ester), or a compound having a similar structure to a fatty acid, such as an epoxidized ether (e.g., epoxidized ether of a fatty acid), or the like. The illustrated molecule for E₃ may be obtained from Hebei Jingu Grease Technology Co., Ltd., of China. Indeed, in certain embodiments, the position of the epoxide, the number of carbons total in the chain of E₃, and the number of carbons between the epoxide of E₃ and the sp2-hybridized carbon of the ester functionality of E₃ may vary, for example between 2 and 10 carbon atoms, and all sub ranges and individual values therebetween. Further, the number of carbons extending from the epoxide functionality in the direction away from the ester functionality may be between 1 and 11, and all sub ranges and individual values therebetween.

As shown in Scheme 7, E₃ may react with D₁ to produce a natural oil branching agent B₁. In particular, E₃ may react with D₁ via an acid-catalyzed ring opening reaction. The acid catalyst may be any suitable type of acid, and, in some embodiments, may be the same type of catalyst as used in the reaction between P₃ and C₂ discussed above. More specifically, the diester functionalized monol D₁ includes multiple carboxylic acid functionalities that are residual from the C₂ molecule (e.g., one residual from the anhydride and one from the unreacted carboxylic acid of the C₂ molecule). Any one or a combination of these carboxylic acid functionalities may be used as the ring-opening nucleophile in the reaction between E₃ and D₁. However, in the depicted reaction, all of the carboxylic acid functionalities react with one molecule of E₃ to produce B₁. Accordingly, in one embodiment, B₁ includes no residual carboxylic acids, and may be considered to be a low molecular weight polyol that is derived from natural oils (e.g., post-consumer recycle natural oils). As noted above, B₁ may have a molecular weight that is between approximately 10% and 60% of the molecular weight of the NOP, between approximately 20% and 55% of the molecular weight of the NOP, or between approximately 25% and 50% of the molecular weight of the NOP (e.g., P₅ discussed below). While the illustrated branching agent B₁ has a molecular weight of approximately 1788, it should be noted that the branching agent B₁ may have a molecular weight of between, for example, 500 and 3,500, such as 1,000 and 3,000, or between 1500 and 2000 (e.g., as varied by any one or a combination of the carbon chain lengths of E₃ and the particular polycarboxylic acid used as C₂). For example, in varying the chain length of E₃, the heptyl (C₇H₁₅) pendant carbon chains of B₁ may be varied (e.g., may be methyl, ethyl, propyl, butyl, pentyl, hexyl, octyl, nonyl, decyl groups), while the connection between the polyether polyol denoted as P₃ and the epoxidized ester E₃ may be varied by varying C₂ (e.g., by modifying the aryl ring of C₂ with one or more additional functionalities).

In accordance with certain embodiments, the reaction between P₃ and C₂ and the reaction between E₃ and D₁ may be performed in a one-pot synthesis. For example, in certain embodiments, all of the reagents P₃, C₂, and E₃, as well as one or more catalysts (e.g., triphenylphosphine, phosphoric acid, and the like) may be charged into a single vessel, heated, and allowed to react for a certain period of time to ultimately produce B₁. Example reaction temperatures and times are discussed in further detail below. However, in a general sense, in embodiments where the reaction Scheme 7 is performed in a one-pot synthesis, the reaction temperature may be between 20° C. and 300° C. (e.g., between 150° C. and 200° C.) and the reaction time may be between 1 minute and 1 day (e.g., between 1 hour and 2 hours).

It should be appreciated that the reaction between E₃ and D₁ may produce, in addition to the desired product in which all of the carboxylic acid moieties have each reacted with a molecule of E₃, other products resulting from, for example, an incomplete conversion of the carboxylic acids (e.g., where not all of the carboxylic acids have reacted). Accordingly, it may be desirable to isolate B₁ from other potential side products. In accordance with present embodiments, such isolation may be performed via extraction (block 64) using one or more solvents.

For example, in embodiments where the reaction performed to produce the branching agent B₁ is carried out at an elevated temperature (e.g., greater than 40° C.), the reaction may be allowed to cool to a predetermined temperature (e.g., less than 40° C., less than 30° C.), and mixed with one or more solvents in order to extract the branching agent B₁. The one or more solvents used for extraction may be any appropriate organic solvent, such as dialkyl ethers, petroleum ether, alkanes, cycloalkanes, and the like. In one embodiment, the solvent may be petroleum ether. In such embodiments, the petroleum ether may remove unreacted E₃ (e.g., epoxidized fatty acid methyl ester) and saturated fatty acid esters. Indeed, in accordance with the present disclosure, the solvent or solvents may include nonpolar, aprotic solvents such as one or more light hydrocarbons (e.g., hydrocarbons having less than 10 carbon atoms, less than 8 carbon atoms, less than 7 carbon atoms, or 6 or less carbon atoms), such as hydrocarbon solvents having between 4 and 8 carbon atoms. While not wishing to be bound by theory, it is believed that the long alkyl chains incorporated from the fatty acid ester (E₃) enables the branching agent B₁ to be soluble/miscible in nonpolar aprotic solvents such as petroleum ether, pentane, hexanes, heptanes, etc., and straight chain, branched, and cyclic versions thereof, such that it is able to be selectively removed from the reaction mixture produced from a one-pot synthesis of Scheme 7. Other solvents are also encompassed by the present technique, such as alcohols (e.g., solvents having between 2 and 10 carbons and a hydroxyl group). Example alcohol solvents that may be used in combination with, or in lieu of, the alkyl solvents noted above include ethanol, propanol (e.g., 1-propanol, 2-propanol), butanol (e.g., n-butanol, t-butanol), and the like.

Upon extraction, the solvent may be removed from the extracted B₁ (extracted from the reaction mixture) by evaporation. By way of example, the solvent (e.g., petroleum ether) may be distilled away from the branching agent B₁ in order to isolate the branching agent B₁. In certain embodiments, the one or more solvents may be recycled and used for subsequent extractions.

It should be noted that the branching technique described herein enables the production of intermediates having a relatively high molecular weight, while also maintaining the viscosity of the intermediates at manageable levels, which can be important for large scale manufacturing processes. Indeed, as discussed above, the branching agent B₁ may be used as a reagent in the transesterification reaction performed according to block 42 of process 26 (FIG. 2), or may be used as a reagent in a different type of reaction, in order to produce an NOP. The branching agent B₁ may have a molecular weight between approximately 1,000 and 10,000, such as between approximately 1% and 40% of the molecular weight of the NOP, between approximately 2% and 35% of the molecular weight of the NOP, or between approximately 5% and 30% of the molecular weight of the NOP.

Thus, after the branching agent B₁ is isolated via extraction in accordance with block 64, the process 60 may include another series of reactions, which are depicted below in Schemes 8 and 9. As shown in Scheme 8 below, the branching agent B₁, after isolation, undergoes transesterification (block 66) with an initiator polyol G₁. The initiator polyol G₁ may be a relatively low molecular weight polyol initiator molecule, such as a polyol derived from sucrose, glucose, or any other natural or synthetic polyols, small chain polyols (e.g., diols, triols), polyols having at least 2 carbons (e.g., between 2 and 20 carbons), branched polyols having at least 2 carbons (e.g., between 2 and 10 carbons), and similar polyol molecules. The initiator polyol, more specifically, may include between 2 and 10 carbons and at least two hydroxyl groups, such as between 2 and 4 hydroxyl groups. In some embodiments, the initiator polyol may include 3 hydroxyl groups and between 2 and 4 carbons. The hydroxyl groups may be primary, secondary, or tertiary, or any combination thereof (e.g., one or more primary hydroxyl groups, and/or one or more secondary hydroxyl groups, and/or one or more tertiary hydroxyl groups).

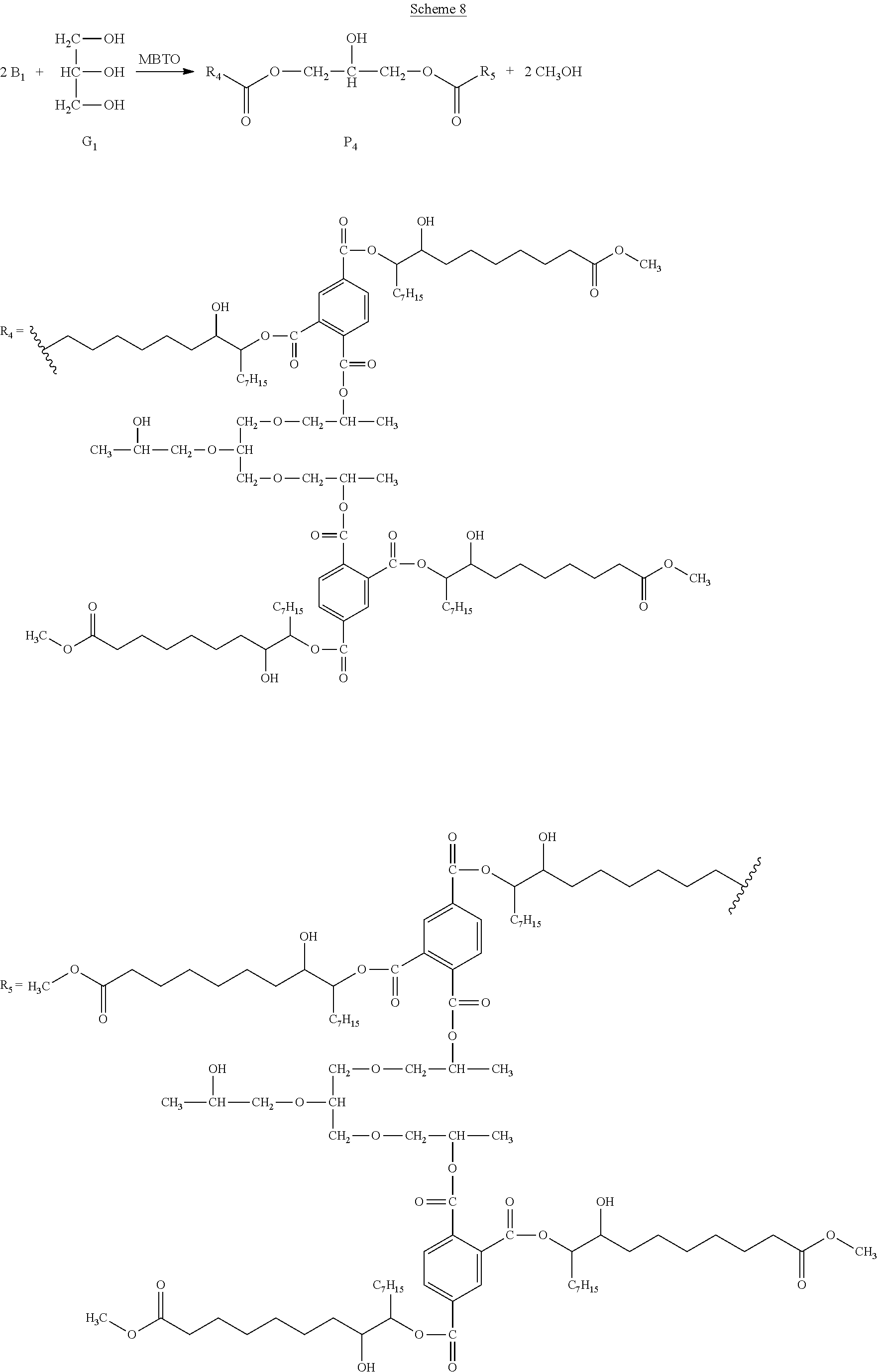

In the illustrated Scheme 8, the initiator polyol is glycerol, which has 3 hydroxyl groups and 3 carbons. The transesterification reaction, as depicted, is facilitated using a catalyst. While any appropriate transesterification catalyst may be used, in some embodiments, the catalyst may be a tin catalyst, such as monobutyl tin oxide (MBTO). By way of non-limiting example, the illustrated reaction between B₁ and G₁ may be performed at a relatively elevated temperature (e.g., between 80° C. and 150° C.) for between 1 hour and 1 day (e.g., between 1 and 2 hours) while under vacuum (e.g., to remove water and drive the reaction forward). For example, the reaction between B₁ and G₁ may be performed for approximately 30 minutes at approximately 95° C., and then for approximately 40 minutes at 120° C.

The transesterification reaction is depicted as using two moles of the branching agent B₁ for every mol of initiator polyol molecule G₁ to produce a first branched polyol P₄. The first branched polyol P₄, as shown in Scheme 8, is the reaction product produced by transesterification of the two primary hydroxyl groups of the polyol initiator G₁ with pendant methyl ethers of the branching agent B₁. As shown, the attachment may occur at the ester in the 2- (ortho) position of the central benzene ring (as in R₄), or may occur at the ester in the 4- (para) position (as in R₅), or both. In one embodiment, as illustrated, P₄ includes reaction at the ester in the ortho position of one molecule of B₁ and reaction at the ester in the para position of another molecule of B1. In other embodiments, the resulting polyol may include a polyol where reaction has occurred only at the para position ester (denoted as P₄′), or only at the ortho position ester (denoted as P₄″), or may include a mixture of any two or more of the illustrated P₄, the para-only molecule P4′, and the ortho-only molecule P₄″.

While not wishing to be bound by theory, it is believed that the first branched polyol P₄ is selectively produced due to steric hindrance of the other hydroxyl and ether functionalities of the branching agent B₁, and the relative availability and enhanced reactivity of the pendant hydroxyl functionalities of the glycerol polyol initiator G₁ (which may, in some embodiments, tautomerize to produce a more active hydroxyl group). However, any number of initiator polyol molecules G₁ may be reacted with B₁ in order to produce a polyol used in accordance with the present technique. Further, in some embodiments, an additional extraction step may be performed after transesterification, for instance using the same types of solvents as set forth above with respect to block 64. Such an extraction may remove unreacted G₁, as well as other residual byproducts.

To enhance the polymerization activity of the first branched polyol P₄, the process 60 may also include capping (block 68) the polyol P₄ with an alkylene oxide (e.g., ethylene oxide, propylene oxide) to produce a capped and branched polyol P₅. An example of this reaction is depicted in Scheme 9 below. By way of non-limiting example, the reaction represented in Scheme 9 may be performed at a temperature of between 100° C. and 150° C. (e.g., 125° C.) and at a pressure of between approximately 0.2 kPa and 0.4 kPa (e.g., 0.3 kPa) for between 1 hour and 10 hours (e.g., 2 hours). The capping performed in accordance with block 68 may also reduce the acid number of the branched polyol P₅, as discussed in further detail below.

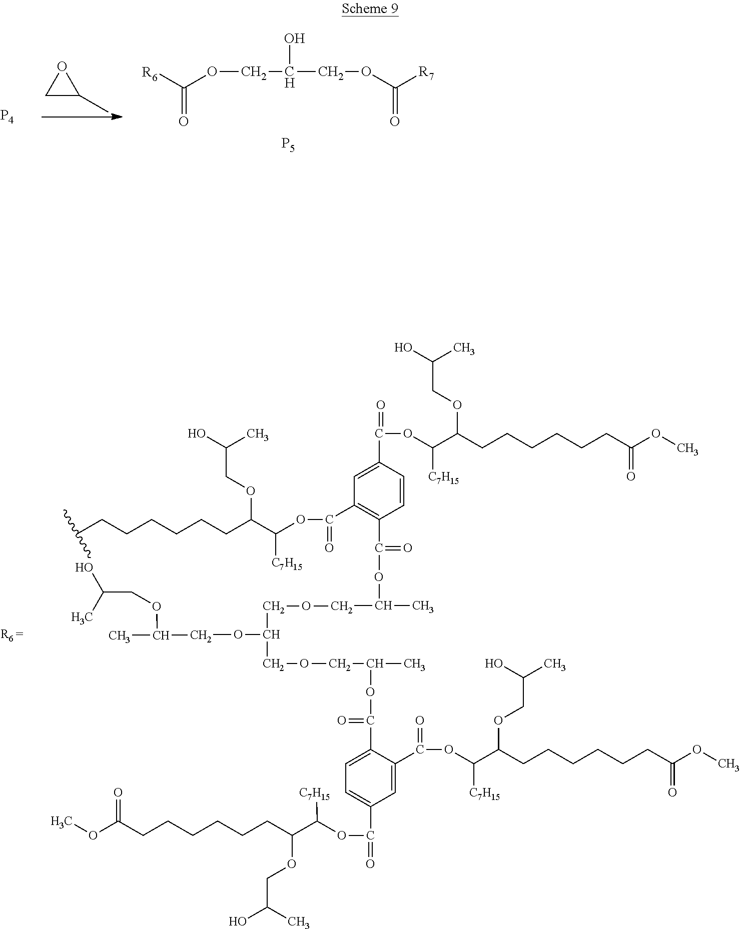

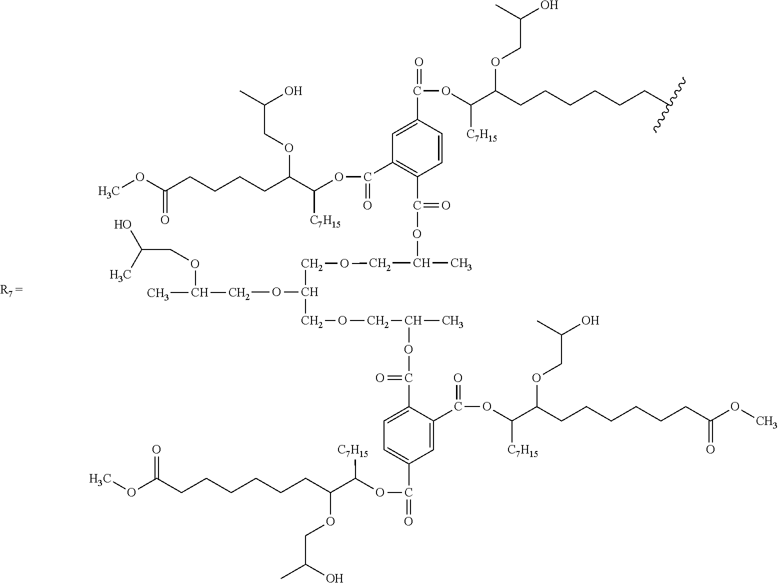

In certain embodiments, the degree of branching of the polyol P₄, while desirable from a molecular weight control standpoint, may reduce the ability of the secondary hydroxyl groups to react with other monomers (e.g., isocyanates) in producing a polyurethane foam. Thus, the alkylene oxide capping may effectively extend the secondary hydroxyl unit away from the sterically crowded polyether portion of the polyol P₅.

It should be noted that while P₅ is shown as only reacting with one mol of propylene oxide (PO) per hydroxyl unit, that in certain embodiments, once a ring-opening is performed at a secondary hydroxyl site to form a new ether linkage, that the PO may begin to polymerize at the site of the original ring opening. Therefore, while illustrated in Scheme 9 as being the opened adduct of PO, P₅ may, in certain embodiments, be a branched polyether having a pendant secondary hydroxyl group. In other words, in some embodiments, the adduct may be an oligomer or a polymer resulting from an oligomerization/polymerization initiated from any one or any combination of the secondary hydroxyl units of P₄.

It should be noted that a number of variations in the reaction schemes presented herein with respect to FIG. 3 may occur to those of skill in the art, such as different catalysts, initiators, reaction times, and so on. Set forth below are a number of experiments run to determine appropriate reaction times, catalysts, polyol initiators, and the like, for the synthesis of the capped and branched polyol P₅.

Each of the experiments below were generally run using the alcohol initiator (e.g., P₃), epoxidized fatty acid methyl ester, trimellitic anhydride (TMA), and different catalysts with the ratios listed below. The particular reagents were charged into a reaction vessel and reacted at temperatures of between 160° C. and 170° C. for between 1 and 1.5 hours. Glycerin (G₁) and monobutyl tin oxide (MBTO) were then introduced to the reaction for 30 min at a temperature of 95° C. under vacuum to produce the branched natural oil polyol P₄.

The effect of the initiator (polyol P₃) is shown in Table 1, where hydroxyl values and epoxy values were determined according the titration methods of Chinese standards GB/T1677-79 and HG/T 2709-95, respectively. As depicted, the hydroxyl values are generally higher with glycol and glycerin as the initiator compared to GY-250. While not wishing to be bound by theory, it is believed that because the glycol and glycerin are relatively shorter, they are more reactive, such that further reaction with the epoxidized oil E₃ is hindered. With increasing amounts of GY-250, the hydroxyl number of P₅ is decreased. Because of the increased amount of TMA ring-opening that occurs as the amount of GY-250 is increased, more carboxyl groups are produced, which may further catalyze the reaction represented by Scheme 7. However, if an excess of GY-250 is used, unreacted, residual GY-250 may cause higher hydroxyl numbers in the final NOP. In Table 1, entry 4 having a GY-250 to TMA weight ratio of 11:17 may be considered to have the most desirable tradeoff between hydroxyl value and epoxy value.

TABLE 1
Effect of Initiator on Hydroxyl Value and Epoxy Value

| | | | | Hydroxyl | Epoxy | |
|---|---|---|---|---|---|---|
| | | Dos- | TMA/ | value/ | value/ | Product |
| Entry | Initiator | age/g | g | (mgKOH/g) | g/100 g | color |
| 1 | Glycol | 4 | 17 | 93.8 | 0.63 | Pale Yellow |
| 2 | Glycerin | 4 | 17 | 96.73 | 0.55 | Yellow |
| 3 | GY-250 | 7 | 17 | 77.55 | 1.24 | Pale Yellow |
| 4 | GY-250 | 11 | 17 | 82.48 | 0.24 | Pale Yellow |
| 5 | GY-250 | 15 | 17 | 96.73 | 0.34 | Pale Yellow |

The effects of different types of catalysts were also examined. As shown in Table 2, different types of catalysts (TEDA (triethylenediamine), H₃PO₄ (phosphoric acid), TPP (triphenylphosphine) and a combination of TPP/H₃PO₄)) were introduced into the ring-opening reaction between D₁ and E₃. As shown by the data in Table 2, when using TEDA as catalyst, the color of the polyol P₅ that is ultimately obtained is dark yellow, which is indicative of impurities. The acid value is under the control with using the strong acid of H₃PO₄. The data in Table 2, in this particular embodiment, indicates that TPP may be considered to be the most desirable catalyst of those listed, for example using the amount listed at entry No. 4.

TABLE 2
Effect of Catalyst on Hydroxyl Value and Epoxy Value

| | | | Hydroxyl | Epoxy | |
|---|---|---|---|---|---|
| | | | value/ | value/ | Product |
| Entry | Catalyst | Dosage/g | (mgKOH/g) | g/100 g | color |
| 1 | TEDA | 0.15 | 83.64 | 0.28 | Dark Yellow |
| 2 | H₃PO₄ | 0.35 | 88.34 | 0.54 | Pale Yellow |
| 3 | TPP/H₃PO₄ | 0.10/0.35 | 86.43 | 0.43 | Pale Yellow |
| 4 | TPP | 0.15 | 82.48 | 0.24 | Pale Yellow |
| 5 | TPP | 0.1 | 88.50 | 0.44 | Pale Yellow |
| 6 | TPP | 0.2 | 82.30 | 0.23 | Pale Yellow |

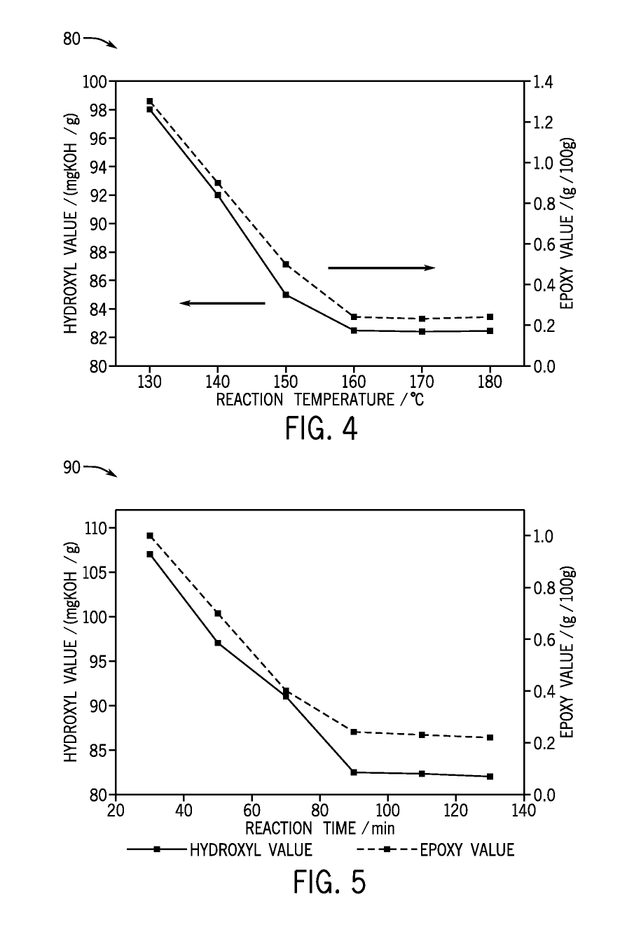

The effects of reaction temperature chosen for the reactions represented in Scheme 7 were also investigated. The results are provided in FIG. 4, which is a combined plot 80 of the hydroxyl number and epoxy number of P₅ as a function of reaction temperature. As shown, the data indicate that at approximately 160° C., the hydroxyl value and epoxy value remain relatively unchanged with increasing temperature up to approximately 180° C. At lower temperatures than 160° C., the hydroxyl value and epoxy value both remain relatively high, which, in this particular embodiment, indicates that approximately 160° C. is a more desirable choice compared to the other listed temperatures under the particular conditions noted above.

The effects of reaction time chosen for the reactions represented in Scheme 7 were also investigated. The results are provided in FIG. 5, which is a combined plot 90 of the hydroxyl number and epoxy number of P₅ as a function of reaction time. As shown, the data indicate that at approximately 90 minutes (1.5 hours), the hydroxyl value and epoxy value remain relatively unchanged with increasing reaction time up to approximately 130 minutes (indicating that the reaction is substantially complete). At shorter reaction times, the hydroxyl value and epoxy value both remain relatively high, which, in this particular embodiment, indicates that approximately 90 minutes is a more desirable choice compared to the other listed reaction times under the particular conditions noted above.

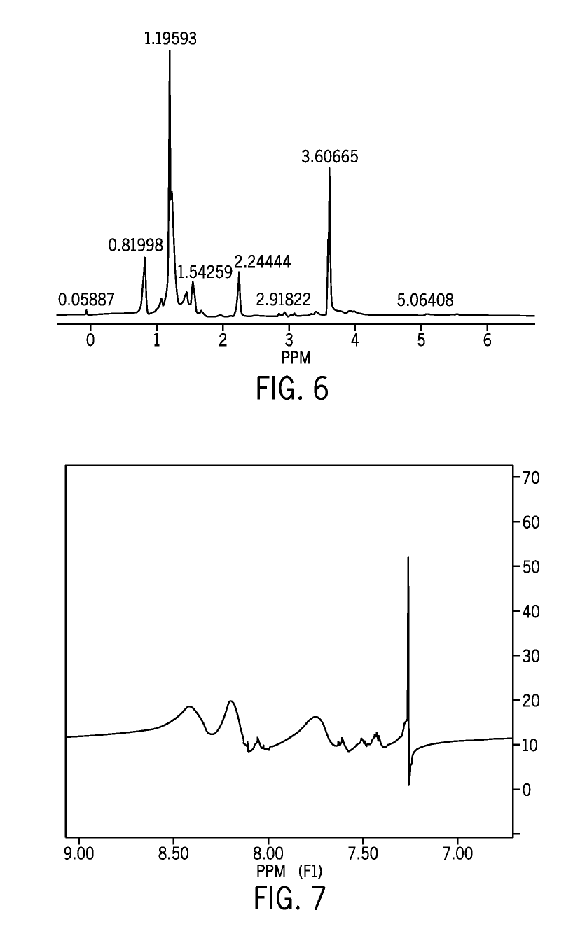

A proton nuclear magnetic resonance (NMR) spectrum (2 regions of the same spectrum), a Fourier transform infra red (FTIR) spectrum, and a gel permeation chromatography (GPC) chromatogram collected on a sample of the capped and branched natural oil polyol (P₅) are provided in FIGS. 6-9, respectively. Specifically, the ¹H NMR spectrum was recorded using Bruker Avance-500 spectrometer (500 MHz) in CDCl₃, and the FT-IR spectrum was recorded in the wavenumber range of 4000-400 cm⁻¹ on a Bruker FT-Vertex-70 spectrophotometer. It should be noted that in addition to the peaks of the ¹H NMR spectrum shown in FIG. 6, the NMR spectrum also includes small peaks indicative of hydrogens from the aryl rings, as shown in FIG. 7, which includes an expanded portion of the same ¹H NMR spectrum, and displaying peaks ranging between δ=7 ppm and δ=8.5 ppm. The GPC chromatogram was obtained using a UV diode array detector and with known molecular weight polystyrene standards to generate a calibration curve. The sample data was reported in terms of the number average (Mn), the weight average (Mw), the polydispersity index (PDI), and the average abundance.

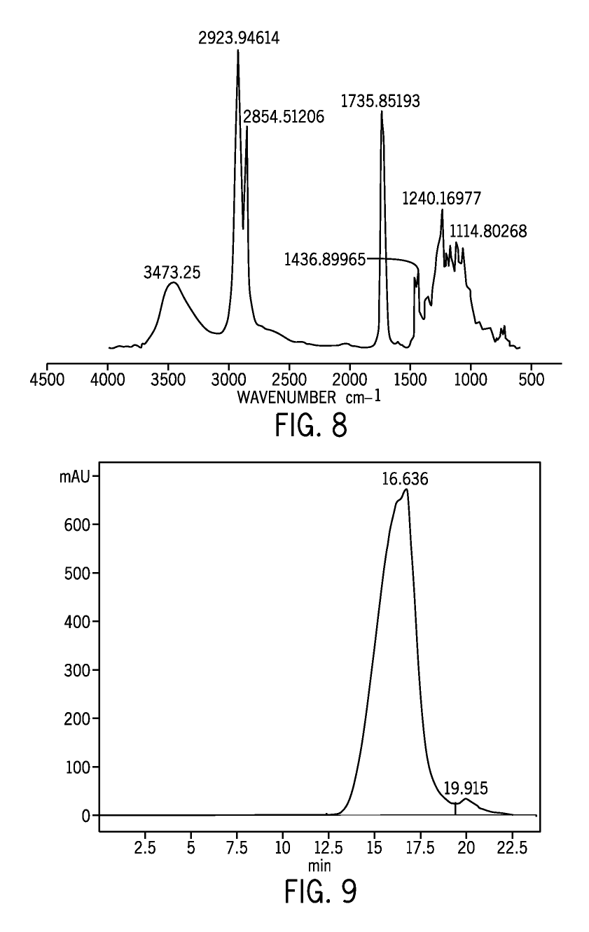

In addition, acid number, hydroxyl number, and water content analyses were performed on a sample of P₅ produced in accordance with the present technique. The molecular weight 1 and molecular weight 2 correspond to the two peaks in the GPC illustrated in FIG. 8. it should be noted that, averaged over 2 runs, the first peak has an average abundance of 97.2%, a Mw of 4,420, and an Mn of 1,970, whereas the smaller peak has an average abundance of 2.4%, a Mw of 88, and an Mn of 57. The water content listed below was obtained through Karl Fischer Titrimetry, and the hydroxyl and acid values were obtained using the USP 36/NF 31 method.

TABLE 3
Example Properties of NOP

| Analysis | Measurement |
|---|---|
| Water Content | 0.145% by weight |
| Hydroxyl Number | 122.01 mg KOH/g |
| Acid Number | 1.88 mg KOH/g |
| Molecular Weight 1 | 1970 |
| Molecular Weight 2 | 57 |
| PDI | 2.25 |

As noted above, the capped and branched polyol P₅, which is a natural oil-derived polyol (e.g., a post-consumer recycle natural oil polyol), may be used in the production of a polyurethane foam in a similar manner as described above with respect to FIG. 1. That is, the capped and branched polyol P₅ may be used as all or a part of the natural oil polyol 28 in FIG. 1. For example, a polyol formulation may be generated using P₅ as the polyol, and, in accordance with an embodiment, may enable the incorporation of up to approximately 40% by weight of natural oil polyol into the polyol formulation used to produce a foam product. By way of example, a flexible, open cell, molded foam product (e.g., polyurethane foam) produced in accordance with present embodiments may incorporate natural oil polyol material (e.g., capped and branched natural oil polyol P₅) in a range of from 5% by weight up to approximately 40% by weight, such as between 10% by weight and 40% by weight, or between 20% by weight and 40% by weight. In some embodiments, the foam's physical properties may not be detrimentally affected, even at higher loadings (e.g., up to 40% by weight) of the capped and branched natural oil polyol P₅. While not wishing to be bound by theory, it is believed that, among other steps, the extraction performed in accordance with block 64 may reduce the presence of unreacted products (e.g., fully saturated acids that are not epoxidized), which can detrimentally affect the physical properties of the foam.

Generally, a polyurethane foam is produced by reacting the post-consumer recycle natural oil polyol, such as P₅ and/or polyol N₂ produced in Scheme 5) with the polyisocyanate formulation. The post-consumer recycle natural oil polyol may be combined with other reactants, such as a blowing agent (e.g., water, volatile organic solvents), a crosslinker, a surfactant, and other additives (e.g., cell openers, stabilizers) to generate a polyol formulation. The polyol formulation may further include other polymeric materials, such as copolymer materials that are configured to impart certain physical properties to the polyurethane foam. Further, in certain embodiments, a catalyst configured to facilitate polyurethane production (i.e., reaction between the hydroxyl groups of the polyol formulation and the isocyanate groups of the isocyanate mixture) may be used, and may be a part of the polyol formulation.

The catalyst may include certain amines (e.g., tertiary amines), amine salts, organometals (e.g., organobismuth and/or organozinc compounds), or other similar catalysts (e.g., combinations or either of a blowing catalyst and a gelling catalyst). Commercial examples of catalysts that may be incorporated into the polyol formulation include DABCO® 33lv amine catalyst (1,4-diazabicyclo[2.2.2]octane) available from Sigma Aldrich Co., LLC of St. Louis, Mo. and BiCAT® bismuth catalysts available from The Shepherd Chemical Company of Norwood, Ohio.

The isocyanate formulation, which is reacted with the polyol formulation, may include one or more different polyisocyanate compounds. Examples of such compounds include methylene diphenyl diisocyanate (MDI), toluene diisocyanate (TDI), or other such compounds having two or more isocyanate groups. The polyisocyanate compounds may also include prepolymers or polymers having an average of two or more isocyanate groups per molecule. The particular polyisocyanate compounds used may depend on the desired end use (i.e., the desired physical properties) of the polyurethane foam.

While only certain features and embodiments of the invention have been illustrated and described, many modifications and changes may occur to those skilled in the art (e.g., values of parameters (e.g., temperatures, pressures, etc.), reactants, used oil source, etc.) without materially departing from the novel teachings and advantages of the subject matter recited in the claims. The order or sequence of any process or method steps may be varied or re-sequenced according to alternative embodiments. It is, therefore, to be understood that the appended claims are intended to cover all such modifications and changes as fall within the true spirit of the invention. Furthermore, in an effort to provide a concise description of the exemplary embodiments, all features of an actual implementation may not have been described (i.e., those unrelated to the presently contemplated best mode of carrying out the invention, or those unrelated to enabling the claimed invention). It should be appreciated that in the development of any such actual implementation, as in any engineering or design project, numerous implementation specific decisions may be made. Such a development effort might be complex and time consuming, but would nevertheless be a routine undertaking of design, fabrication, and manufacture for those of ordinary skill having the benefit of this disclosure, without undue experimentation.

1. An open cell, molded foam produced by a process, the process comprising:
reacting a polyol formulation with an isocyanate mixture, wherein at least one component of the polyol formulation is produced by a process comprising:
reacting a first polyol with an acid anhydride compound to produce a monol having two or more polycarboxylic acid substituents;
reacting the monol having two or more polycarboxylic acid substituents with an epoxidized fatty acid of a post-consumer recycle oil to produce a polyol branching agent;
reacting the polyol branching agent with a polyol initiator to link at least two molecules of the polyol branching agent together to produce a branched polyol; and
capping the branched polyol with an alkylene oxide to produce a natural oil-based polyol having a molecular weight of between 2,000 and 6,000.

2. The foam of claim 1, wherein the process used to produce the foam further comprises extracting the polyol branching agent from a reaction mixture produced from the monol having two or more polycarboxylic acid substituents and the epoxidized fatty acid of a post-consumer recycle oil using one or more solvents.

3. The foam of claim 2, wherein the one or more solvents comprise a hydrocarbon solvent having between 2 and 8 carbon atoms.

4. The foam of claim 2, wherein the one or more solvents comprise petroleum ether, and the extraction separates the polyol branching agent from saturated fatty acid components of the reaction mixture, and wherein the extraction is performed before the polyol branching agent is reacted with the polyol initiator.

5. The foam of claim 1, wherein the first polyol is a glycerin-initiated polyol based on propylene oxide.

6. The foam of claim 5, wherein the first polyol has a molecular weight of between 200 and 300.

7. The foam of claim 1, wherein the acid anhydride compound comprises an aryl acid anhydride having at least one carboxylic acid.

8. The foam of claim 1, wherein reacting the monol having two or more polycarboxylic acid substituents with the epoxidized fatty acid of a post-consumer recycle oil comprises performing a catalyzed ring-opening of the epoxidized fatty acid using the monol having two or more polycarboxylic acid substituents.

9. The foam of claim 1, wherein the polyol branching agent has a molecular weight that is between approximately 25% and 50% of a molecular weight of the natural oil-based polyol.

10. The foam of claim 1, wherein the natural oil-based polyol has a polydispersity index (PDI) of between approximately 1.5 and 2.5, wherein the PDI is defined as the ratio of the weight-average molecular weight, Mw, to the number-average molecular weight, Mn.

11. The foam of claim 1, wherein between approximately 10% by weight and 40% by weight of the foam is from the natural oil-based polyol.

12. A method, comprising:
reacting a first polyol with an acid anhydride compound to produce a monol having two or more polycarboxylic acid substituents;
reacting the monol having two or more polycarboxylic acid substituents with an epoxidized fatty acid of a post-consumer recycle oil to produce a polyol branching agent;
extracting the polyol branching agent from a reaction mixture produced from the monol having two or more polycarboxylic acid substituents and the epoxidized fatty acid of a post-consumer recycle oil using one or more solvents;
reacting the extracted polyol branching agent with a polyol initiator to link at least two molecules of the polyol branching agent together to produce a branched polyol; and
capping the branched polyol with an alkylene oxide to produce a natural oil-based polyol having a molecular weight of between 2,000 and 6,000.

13. The method of claim 12, wherein the one or more solvents comprise a hydrocarbon solvent having between 2 and 8 carbon atoms.

14. The method of claim 12, wherein the first polyol is a glycerin-initiated triol based on propylene oxide and having a molecular weight between 200 and 300.

15. The method of claim 12, wherein the polyol branching agent has a molecular weight of between 1,000 and 2,000.

16. The method of claim 12, wherein the polyol branching agent has a molecular weight that is between approximately 25% and 50% of a molecular weight of the natural oil-based polyol.

17. The method of claim 12, wherein:
the first polyol comprises a compound having the formula: 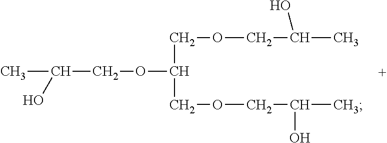 [SMILES: CC(O)COCC(COCC(C)O)OCC(C)O]
and
the acid anhydride is an aryl anhydride having at least one carboxylic acid.

18. The method of claim 12, wherein the polyol branching agent is produced in a one-pot synthesis in which the first polyol is reacted with the acid anhydride and the monol having two or more polycarboxylic acid substituents is reacted with the epoxidized fatty acid of a post-consumer recycle oil.

19. The method of claim 12, wherein the natural oil-based polyol has a polydispersity index (PDI) of between approximately 1.5 and 2.5, wherein the PDI is defined as the ratio of the weight-average molecular weight, Mw, to the number-average molecular weight, Mn.

20. An open cell, molded polyurethane foam comprising the reaction product of a reaction mixture comprising:
an isocyanate mixture; and
a polyol formulation, comprising a glycerin-initiated, alkylene-oxide capped natural oil polyol having a molecular weight of between 3,000 and 8,000 and a polydispersity index (PDI) of between approximately 1.5 and 2.5, wherein the PDI is defined as the ratio of the weight-average molecular weight, Mw, to the number-average molecular weight, Mn.

21. A method, comprising:
reacting a plurality of refined oils produced from used oils with an alcohol to generate a plurality of modified oils;
oxidizing the plurality of refined oils to generate a plurality of epoxidized oils; and
reducing the plurality of epoxidized oils to generate a plurality of polyhydroxylated oils; and
producing a plurality of natural oil polyols from the plurality of natural oil polyols, wherein the plurality of natural oil polyols comprise primary alcohols and have a molecular weight of at least 3,000.

22. The method of claim 21, wherein the plurality of refined oils comprise a plurality of triglycerides having the formula: 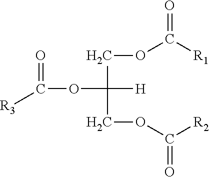
wherein R1, R2, and R3 independently comprise an unsaturated or saturated carbon chain having between 16 and 22 carbon atoms.

23. The method of claim 22, wherein R1, R2, and R3 independently comprise a stearic, elaidic, linoleic, α-linolenic, or α-eleostearic substituent.

24. The method of claim 21, wherein the alcohol comprises glycerol, sucrose, pentaerythritol, or a combination thereof.

25. The method of claim 21, wherein the plurality of modified oils comprise a glyceride having the formula: 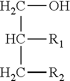
wherein R1 and R2 comprise hydroxyl or aliphatic moieties, and the aliphatic moieties comprise stearate, elaidate, linoleate, α-linolenate, α-eleostearate.

26. The method of claim 25, wherein R1 or R2 comprise a hydroxyl group.

27. The method of claim 21, wherein the plurality of epoxidized oils comprise an epoxidized glyceride.

28. The method of claim 21, wherein the plurality of polyhydroxylated oils comprise a polyhydroxylated glyceride with a plurality of secondary hydroxyl groups.

29. The method of claim 28, wherein the plurality of secondary hydroxyl groups are reacted with an extender to form the plurality of natural oil polyols.

30. The method of claim 29, wherein the extender comprises ethylene oxide or propylene oxide.

31. The method of claim 30, wherein the plurality of natural oil polyols comprise a polyhydroxylated glyceride with a plurality of primary hydroxyl groups.

32. The method of claim 31, wherein the molecular weight of the plurality of natural oil polyols is between 3,000 and 8,000.

33. A method, comprising:
treating a plurality of used oils to form a plurality of refined oils;
subjecting the plurality of refined oils to a process that generates a plurality of modified oils; and
reacting the plurality of modified oils to produce a plurality of polyhydroxylated oils; and
producing a plurality of natural oil polyols from the plurality of polyhydroxylated oils, wherein the plurality of natural oil polyols comprise primary alcohols and have a molecular weight of at least 3,000.

34. The method of claim 33, wherein the plurality of used oils comprise animal oil, cooking oil, or a combination thereof.

35. The method of claim 33, wherein the plurality of refined oils comprise a plurality of triglycerides having the formula: 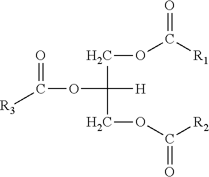
wherein R1, R2, and R3 independently comprise an unsaturated or saturated carbon chain having between 8 and 22 carbon atoms.

36. The method of claim 35, wherein R1, R2, and R3 independently comprise a stearic, elaidic, linoleic, α-linolenic, or α-eleostearic substituent.

37. The method of claim 33, wherein the plurality of polyhydroxylated oils comprise a polyhydroxylated glyceride with a plurality of secondary hydroxyl groups.

38. The method of claim 33, wherein the plurality of natural oil polyols comprise a polyhydroxylated glyceride with a plurality of primary hydroxyl groups.

39. The method of claim 33, wherein the plurality of natural oil polyols have a repeating unit having the following formula: 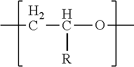
wherein R is hydrogen or methyl substituent.

40. The method of claim 33, wherein the molecular weight of the plurality of natural oil polyols is between 3,000 and 8,000.

41. A foam produced by a process comprising:
reacting a polymeric polyol formulation with an isocyanate mixture, wherein one component of the polymeric polyol formulation is produced by a process comprising:
reacting a plurality of refined oils produced from used oils with an alcohol to generate a plurality of modified oils; and
oxidizing the plurality of refined oils to generate a plurality of epoxidized oils;
reducing the plurality of epoxidized oils to generate a plurality of polyhydroxylated oils; and
producing a plurality of natural oil polyols from the plurality of polyhydroxylated oils, wherein the plurality of natural oil polyols comprise primary alcohols and have a molecular weight of at least 3,000.

42. The foam of claim 41, wherein the plurality of refined oils comprise a plurality of triglycerides having the formula: 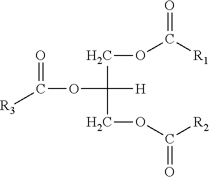
wherein R1, R2, and R3 independently comprise an unsaturated or saturated carbon chain having between 8 and 22 carbon atoms.

43. The foam of claim 42, wherein R1, R2, and R3 independently comprise a stearic, elaidic, linoleic, α-linolenic, or α-eleostearic substituent.

44. The foam of claim 42, wherein the plurality of natural oil polyols comprise a polyhydroxylated glyceride with a plurality of primary hydroxyl groups.

45. The foam of claim 42, wherein the molecular weight of the plurality of natural oil polyols is between 3,000 and 8,000.
